(19) United States
(12) Patent Application Publication
(10) Pub. No.: US 2011/0259973 A1
(43) Pub. Date: Oct. 27, 2011

(54) Toilet flush activated air freshener

(76) Inventors: Ernest A. Pedicano, Valhalla, NY (US); Adam D. Straus, (US)

(21) Appl. No.: 13/066,613

(22) Filed: Apr. 19, 2011

Related U.S. Application Data
(60) Provisional application No. 61/327,825, filed on Apr. 26, 2010.

(51) Int. Cl. A61L 9/04

(52) U.S. Cl. 239/44

(57) ABSTRACT

A machine for dispensing an air freshener spray in a bathroom and, more specifically, to dispensing such by the act of flushing a toilet with use of a floatation switch in the toilet tank water that activates a device upon water levels dropping, a device may generally be one which plugs directly into an electrical outlet, one that operates remote from the outlet and is powered by batteries, the device may plug directly through a cord into an electrical outlet, the device may be powered by batteries, these devices may operate with a cartridge containing a fragrance, the cartridge may be refillable, the fragrance may be a liquid, the fragrance may be a gel, the fragrance may be released by atomization, vaporization of fragrance may occur due to heating (evaporation), the fragrance cartridge may include a wick, fragranced liquid may be drawn up wick and evaporate on exposure to air. In a second embodiment, the activating device may be attached to the lid of the seat of the toilet.

### CROSS REFERENCE TO RELATED APPLICATIONS

This application is based on provisional application Ser. No. 61/327,825, filed on Apr. 26, 2010.

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of air care and more specifically to a machine for dispensing of an air freshener spray in a bathroom and, more specifically, to dispensing such by the act of flushing a toilet. Air freshener devices are relatively commonplace, particularly in domestic environments where they may be used to create a pleasant ambience or to mask unwanted odors. The device may generally be one which plugs directly into an electrical outlet or one that operates remote from the outlet and is powered by batteries or through a cord which plugs into an electrical outlet. Either of these devices may operate in combination with a cartridge containing a fragrance. The refillable cartridge may contain a gel which includes a fragrance and, upon contact with air, the fragrance is released from the gel due to atomization. The refillable cartridge may include a wick, where a fragranced liquid may be drawn up the Either of these devices may operate in combination with a cartridge containing a fragrance. The refillable cartridge may contain a gel which includes a fragrance and, upon contact with air, the fragrance is released from the gel due to atomization. The refillable cartridge may include a wick, where a fragranced liquid may be drawn up the wick and evaporate on exposure to the air. Vaporization may occur due to spraying or from heating (evaporation). The need for odor control is synonymous with the use of the toilet. Most odors that arise from toilets become quickly distributed above and about the area of the toilet making fragrance dispensing in the immediate area of the toilet fairly effective in masking the odors before they circulate throughout the bathroom. In some cases the odor control chemistry may mask the odor with a stronger scent, in other cases it may contain ingredients designed to combat bacteria. In any event, there is a need to distribute the fragrance as much as possible in as short a time as possible in order to defeat the odors. Accordingly, it may be desirable to place an odor control device near the toilet itself and activate the device coincident with use of the toilet, such as upon flushing. Having the dispensing device adjacent the source of the odors in addition to more effective management of the odors may also minimize the length of any connecting devices between the dispenser and the activating mechanism.

With regard to remote activation of an air freshener due to the change in level in a toilet tank, attention is directed to United States Application Publication No. 2007/0204388 which was filed on Mar. 6, 2006 and is entitled “Automated Remote Bathroom Air Freshener”. The allowed claims are directed at a remote dispenser that is mounted to the ceiling of a bathroom, and can rotate to spray a surrounding area and includes a reservoir, spray pump, nozzle, drive motor and a signal receiver. A sending unit is mounted in the toilet tank, including an antenna, a transmitter, a battery, a level sensor and a float. The float includes a proximity sensor and responds to the water level as it moves up and down by moving up and down a shaft and in doing so causes the transmitter to emit a wireless signal to the remote dispenser. The signal actuates the drive motor and pump for a set period of time. The reference does not disclose a solenoid or a switch that tilts with the change in water level. U.S. Pat. No. 4,358,860 issued on Nov. 16, 1982 and is entitled “Automatic Actuator For Air Freshener Dispenser Or The Like For Toilets”. It appears to be directed at a cup that rises and descends with fluctuations in the water level and pivots an actuator arm to automatically dispense a predetermined amount of freshener.

United States Application Publications No. 2008/0272208 entitled “Spraying Device” and No. 2008/0279731 entitled “Device” disclose a spraying device for sanitizing air which includes a solenoid.

Prior technology lack the qualities and benefits of an apparatus and method for dispensing of an air freshener spray in a bathroom to dispense by the act of flushing a toilet.

### BRIEF SUMMARY OF THE INVENTION

The primary advantage of the invention is an apparatus and method for dispensing of an air freshener spray in a bathroom to dispense by the act of flushing a toilet.

Another advantage of the invention is the act of flushing a toilet, which activates the fragrance delivery device to dispense fragrance or odor combating chemicals.

Another advantage of the invention is to place an odor control device near the toilet itself and activate the device coincident with use of the toilet, such as upon flushing.

A further advantage of the invention is having the dispensing device adjacent the source of the odors in addition to more effective management of the odors may also minimize the length of any connecting devices between the dispenser and the activating mechanism.

Yet another advantage of the invention is the need to distribute the fragrance as much as possible in as short a time as possible in order to defeat the odors.

Still yet another advantage of the invention is no tools are needed for installation.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a machine for dispensing of an air freshener spray in a bathroom and, more specifically, to dispensing such by the act of flushing a toilet comprising: use of a floatation switch in the toilet tank water that activates the device upon a water level drop, a device may generally be one which plugs directly into an electrical outlet or one that operates remote from the outlet and is powered by batteries, the device may plug directly through a cord which plugs into an electrical outlet, the device may be powered by batteries, these devices may operate in combination with a cartridge containing a fragrance, the fragrance cartridge may be refillable, the fragrance may be a liquid, the fragrance may be a gel, the fragrance may be released upon contact with air due to atomization, the fragrance cartridge may include a wick, fragranced liquid may be drawn up the wick and evaporate on exposure to the air, and vaporization of fragrance may occur due to spraying or from heating (evaporation).

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

Various features of the present invention will now become readily apparent to those skilled in the art from the following detailed description, wherein the invention is shown along with certain preferred embodiments. As will be realized the invention is capable of other and different embodiments, and its several details are capable of modification in various respects. Accordingly, the description is to be regarded as illustrative in nature and not as restrictive. In a first exemplary embodiment, the activating device, for instance a tilt switch, or a buoyant floating switch may be encased in a water-proof cover and disposed so as to float adjacent the surface of the water in the tank of a toilet. Upon flushing the toilet, the switch tips changes attitude or position due to the receding water level and activates a valve such as a solenoid in a fragrance dispensing device. In a second exemplary embodiment, the tilt switch may be attached to the toilet seat lid such that lowering the lid activates the switch.

FIG. 1 is a schematic illustration of a toilet tank 10 such as is known in the art with the front wall removed for clarity. The tank 10 may have an interior 12 for selectively holding flushing water 14. A fill unit 16 for controlling water flow from a water supply to the tank is mounted in the tank. A lever 18, coupled to a float 20, controls the fill valve 22. A flushing mechanism in the form of a flapper valve 24 empties the water in the tank into a tank outlet 26. The tank outlet is in fluid connection with a toilet bowl (not shown). A filler tube 28 in communication with the fill valve 22, supplies water from the fill unit 16 into the overflow pipe 30. Water dispensed from the overflow pipe 30 proceeds through the flushing mechanism 24 and tank outlet 26 to the bowl (not shown) upon demand (by actuation of the linking mechanism 32 by pressing upon the handle 34). The tank 10 further includes an exemplary activating device in the form of, for instance, a tilt switch 50, preferably encased in a water-proof cover and disposed so as to, for instance, float on the surface 15 of the water 14 in the tank 10 of a toilet. As shown, the switch 50 further includes a cord 52 capable of being plugged into a dispensing device 60 (see FIG. 2). It is contemplated that this could also be a wireless connection. The switch may be disposed to float adjacent the surface of the water 14 in such a manner that the switch 50 is in a substantially horizontal position when the tank 10 has been flushed and refilled (as shown). A clip 56 may be provided to retain the cord 52 in a given position such that the switch changes attitude from substantially horizontal floating on the water in the tank to substantially vertical hanging above the water upon flushing of the toilet. In a related embodiment, the switch 50 may be a ball switch and may be mounted on the linking mechanism 32 or handle 34 and be tipped when the toilet is flushed and completes an electrical circuit. The tilt switch 50 may be a ball switch which allows or interrupts the flow of electric current in an electrical circuit in a manner that is dependent on the switch's physical position or alignment relative to the pull of gravity. Such a switch may include one or more sets of electrical contacts sealed in an envelope which contains the ball. Gravity pulls the ball and allows it to drop to the lowest point in the envelope. When the switch is tilted in the appropriate direction, the ball touches a set of contacts, thus completing the electrical circuit through those contacts. Tilting the switch the opposite direction causes the ball to move away from that set of contacts, thus breaking that circuit. When the tank level rises, the switch may resume the earlier attitude. It is contemplated that other types of switches may be used to activate the spray, such as a motion switch, a mercury switch or a magnetic reed switch that is connected to a magnet that floats on the surface of the water in the tank. Movement of the magnet to a second position due to the change in level of the water activates the switch and trigger the electrical circuit. In other words, a bracket that extends from the edge of the tank may support a buoyant magnet adjacent the surface of the water on the inside of the tank, and may also support the dispensing device and a magnetic reed switch on the outside of the tank. Movement of the magnet due to changes in the water level in the tank may activate the reed switch. Further, the switch may be a proximity switch that can sense the water level from outside the tank, for instance, through the tank wall, and activate the dispenser.

FIG. 2 is a perspective view of the dispensing device 60 of this disclosure placed in proximity to a toilet 100 including a tank 10 containing the activating device. The toilet 100 includes a base 102 having a bowl 104, a seat 106 having a lid or cover 108 that can be raised and lowered, and a flush tank 10. The tank may be equipped with a flushing handle 34. Disposed on the top of the tank 10 is a dispensing device 60 which contains materials for fragrance applications, deodorizing and/or sanitizing the air in the immediate vicinity of the toilet 100. The dispensing device 60 may include a socket 62 into which a connector 54 on the end of the cord 52 may be electrically connected. It is further contemplated that the dispenser may be at any location adjacent the toilet including but not limited to hanging from or mounted on the toilet or on a wall bracket.

FIG. 3A is a front view of the dispensing device 60.

FIG. 3B is a cross-sectional view of the dispensing device of FIG. 3A taken along lines A-A, illustrating placement of a circuit board 64 for controlling the device, the board further including a timing circuit (not shown). Also shown is a plug connector 62 for attaching to the connector 54 on the end of the cord 52.

FIG. 3C is a cross-sectional view of the dispensing device of FIG. 3B taken along lines B-B, illustrating a battery pack 66 for providing power to the device and a 5 solenoid 68 for activating an aerosol can 70 containing the fragrance under pressure. The fragrance may be sprayed into the air though nozzle 72 which may be vertically disposed. It is contemplated that the dispensing device may include a fan or mini-pump and atomizer for dispersal of the fragrance instead of an aerosol. The container 60 may further include a plastic housing 63. It is further contemplated that the battery pack may be replaced by a solar cell which is part of an electrical circuit that includes a capacitor, which may be charged by the solar cell. When the switch is activated, the capacitor will discharge and activate a solenoid which causes the aerosol to spray. In addition, the dispenser may include a small generator which the user can crank every few days to change an on-board capacitor to provide the necessary power.

FIG. 4 is a schematic illustration of the toilet tank of FIG. 1 illustrating the position of the activating device 50 upon flushing of the toilet and activation of the dispensing device. The change in water level in the tank causes a change in attitude (angle) or position of the activating device and completes the circuit in the switch signaling the dispenser to spray a fragrance into the air.

FIG. 5 is a partial side view of the toilet of FIG. 2 including the dispensing device 60 with the activating device 50A attached to the toilet seat lid or cover 108 by a clip 66. In somewhat similar fashion to the tilt switch used in the tank, a switch 50A may be used here to sense whether the lid 108 is up or down and send a signal to activate the dispenser when the lid is placed down, changing the attitude of the switch on the seat 106.

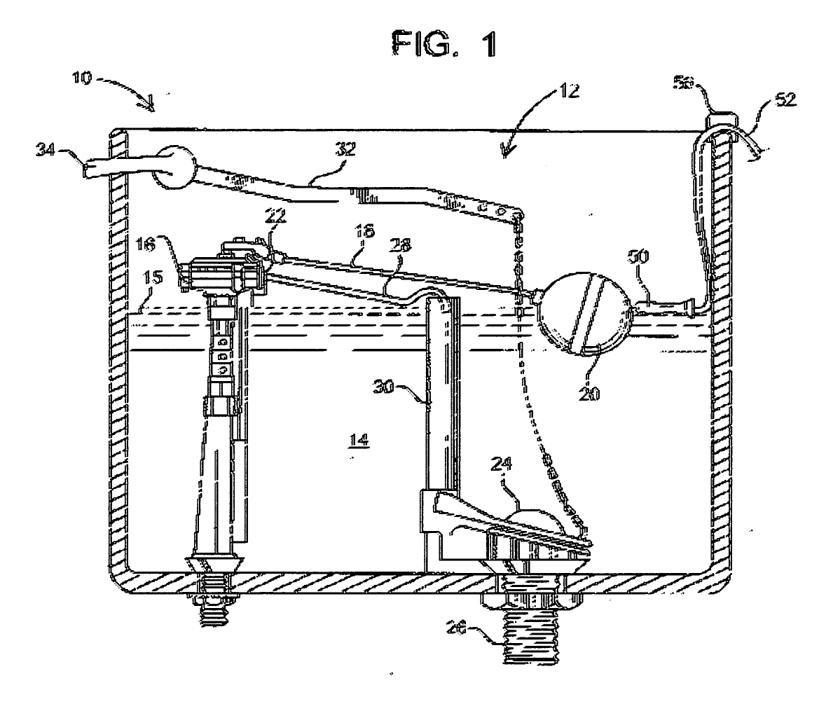

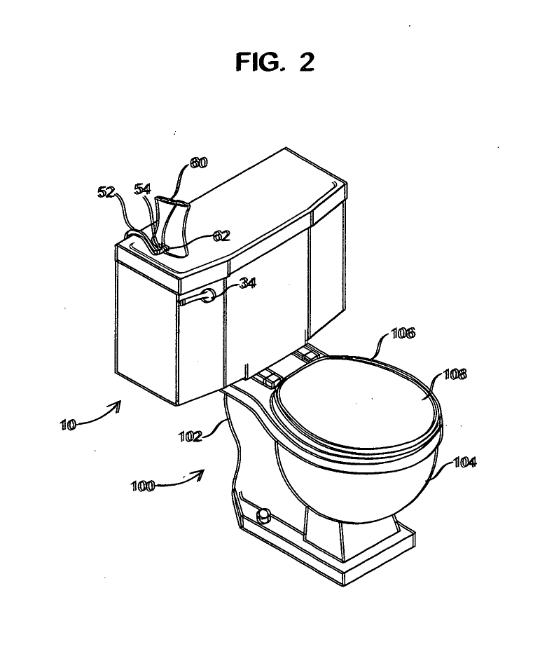

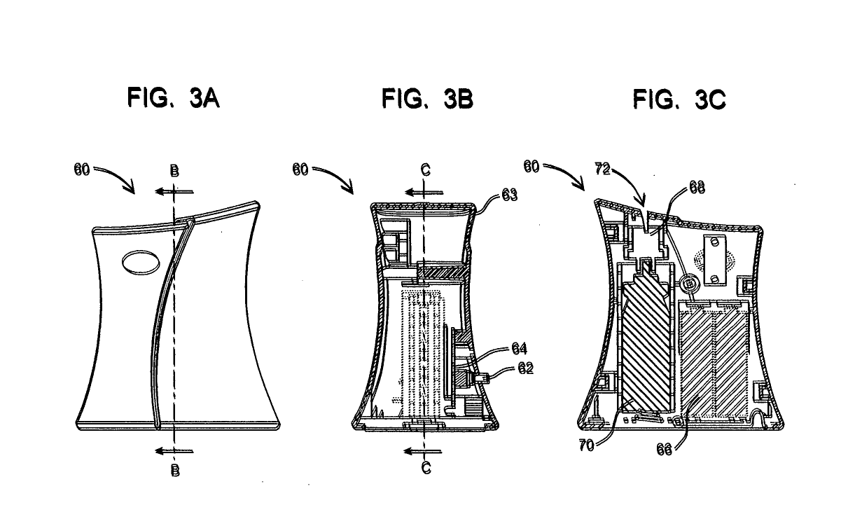

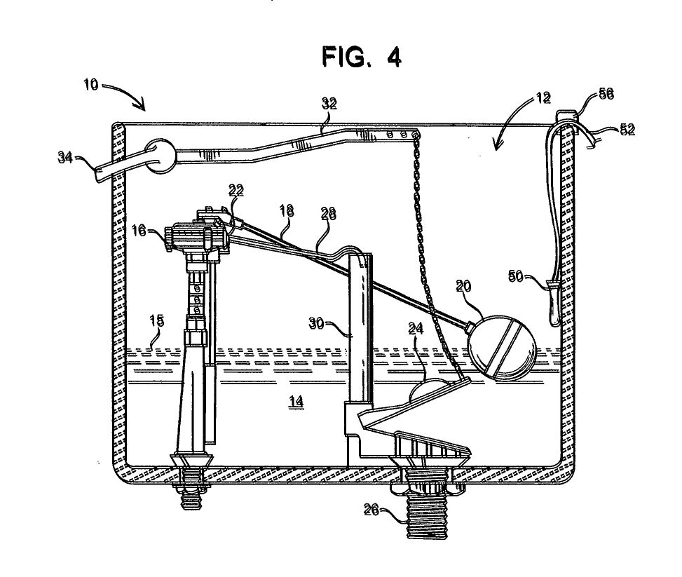

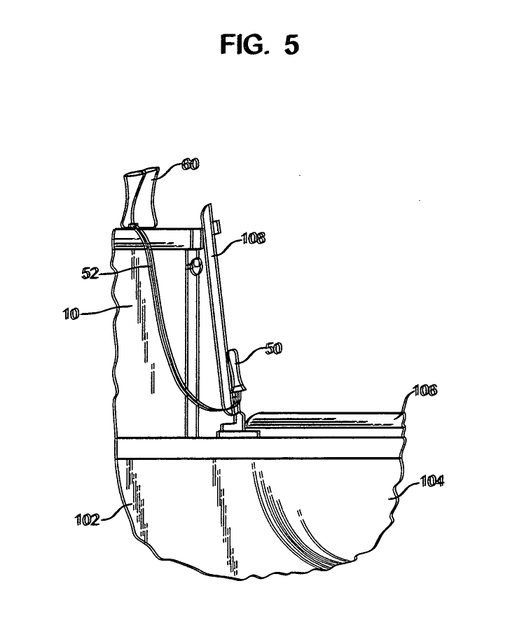

In addition, it is contemplated that the fragrance in liquid form may be dispensed onto a fibrous sheet, such as felt, mounted on the outside of the dispenser, and that the fragrance may relatively slowly be released by or evaporate from the sheet. The timing circuit may be “smart” and apply more fragrance, such as by spraying for a longer time, by taking a sample of the air upon activation of the switch and comparing the reading to a predetermined value taken when the device has not been activated.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

An apparatus and method for dispensing of an air freshener spray in a bathroom to dispense by the act of flushing a toilet. The act of flushing a toilet, activates the fragrance comprising a delivery device to dispense fragrance or odor combating chemicals. To place an odor control device near the toilet itself and activate the device coincident with use of the toilet, such as upon flushing. Having the dispensing device adjacent the source of the odors in addition to more effective management of the odors may also minimize the length of any connecting devices between the dispenser and the activating mechanism. There is a need is to distribute the fragrance as much as possible in as short as time as possible in order to defeat the odors no tools needed for installation.

Use of a floatation switch in the toilet tank water that activates device upon water level drop, a device may generally be one which plugs directly into an electrical outlet or one that operates remote from the outlet and is powered by batteries, the device may plug directly through a cord which plugs into an electrical outlet, the device may be powered by batteries, use of a switch to activate the device outside of toilet tank may use a proximity sensor or other type sensor that can sense a change in water level from out side tank or through the tank wall, the device may use a proximity switch hung down interior of toilet tank to know change of water level, using the water in toilet tank as the battery to power device by using an anode and diode placed separately in tank along with a tablet made out of an acid and placed in tank to make the water acidic, so creating the chemical reactions needed to generate electricity, the device may be powered by a generator that the user cranks to charge a capacitor like component in the device, the device may use a solar cell to collect electricity to power and trigger device, device may use crystals applied to toilet seat, when sat upon the resulting compression of crystals creates a electrical charge that is used to power and trigger device. These devices may operate in combination with a cartridge containing a fragrance, the fragrance cartridge may be refillable, the fragrance may be a liquid, the fragrance may be a gel, the fragrance may be released upon contact with air due to atomization the fragrance cartridge may include a wick, fragranced liquid may be drawn up the wick and evaporate on exposure to the air, vaporization of fragrance may occur due to spraying or from heating (evaporation).

The odor control chemistry of the fragrance may mask the odor with a stronger scent, the fragrance may contain ingredients designed to combat bacteria, the fragrance in liquid form may be dispensed onto a fibrous sheet, such as felt, the felt may be mounted on the outside of the dispenser, the fragrance may relatively be slowly released by evaporation from the sheet. The timing circuit may be “smart” and apply more fragrance, such as by spraying, and for a longer time.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. A machine for dispensing of an air freshener spray in a bathroom and, more specifically, to dispensing such by the act of flushing a toilet comprising:
use of a floatation switch in the toilet tank water that activates device upon water level drop;
a device may generally be one which plugs directly into an electrical outlet or one that operates remote from the outlet and is powered by batteries;
the device may plug directly through a cord which plugs into an electrical outlet;
use of a switch to activate the device outside of toilet tank may use a proximity sensor or other type sensor;
use of a proximity switch hung down interior of toilet tank to know change of water level;
the activating switch for the device may be attached to the lid of the seat of the toilet;
using the water in toilet tank as the battery to power device by using an anode and diode placed separately in tank along with a tablet made out of an acid, placed in tank to make the water acidic, so creating the chemical reactions needed to generate electricity;
the device may be powered by a generator that the user cranks to charge a capacitor like component in the device;
the device may use a solar cell to collect electricity to power and trigger device;
device may use crystals applied to toilet seat, when sat upon the resulting compression of crystals creates a electrical charge that is used to power and trigger device;
the device may be powered by batteries;
these devices may operate in combination with a cartridge containing a fragrance;
the fragrance cartridge may be refillable;
the fragrance may be a liquid;
the fragrance may be a gel;
the fragrance may be released upon contact with air due to atomization;
the fragrance cartridge may include a wick, fragranced liquid may be drawn up the wick and evaporate on exposure to the air; and
vaporization of fragrance may occur due to spraying or from heating (evaporation).
